(19) United States
(12) Patent Application Publication
(10) Pub. No.: US 2015/0202228 A1
(43) Pub. Date: Jul. 23, 2015

(54) SLEEP-IMPROVING AGENT, NON-REM SLEEP TIME-INCREASING AGENT, AND SEDATIVE AGENT

(71) Applicant: FUJIFILM CORPORATION, Tokyo (JP)

(72) Inventors: Hitomi SAITO, Kanagawa (JP); Yuichi OHASHI, Kanagawa (JP)

(21) Appl. No.: 14/673,878

(22) Filed: Mar. 31, 2015

Related U.S. Application Data
(63) Continuation of application No. PCT/JP2013/076730, filed on Oct. 1, 2013.

(30) Foreign Application Priority Data
Oct. 3, 2012 (JP) 2012-221752

(51) Int. Cl. A61K 33/30; A61K 31/122; A61K 36/064; A61K 31/315

(52) U.S. Cl. CPC A61K 33/30; A61K 31/315; A61K 31/122; A61K 36/064

(57) ABSTRACT

A sleep-improving agent, a non-REM sleep time-increasing agent, and a sedative agent, each of which includes a lipid-soluble antioxidant and a divalent metal as active ingredients.

### CROSS-REFERENCE TO RELATED APPLICATIONS

This application is a continuation application of International Application No. PCT/JP2013/076730, filed Oct. 1, 2013, which is incorporated herein by reference. Further, this application claims priority from Japanese Patent Application No.2012-221752, filed Oct. 3, 2012, which is incorporated herein by reference.

### TECHNICAL FIELD

The present invention relates to a sleep-improving agent, a non-REM sleep time-increasing agent, and a sedative agent.

### BACKGROUND ART

In modern society, the number of people suffering from insomnia is increasing year by year. Possible causes of insomnia include various physiological and psychological factors. It is said that cases of insomnia caused by psychological factors have tended to increase in recent years. Examples of the causes of insomnia include decreased balance-recovering capacity of autonomic nerves due to various cares and worries which occur in a stressful society.

Thus, in current social environments in which psychological stress is increasing, a composition having an excellent sleep-promoting effect and sedating effect is in demand.

Sleep includes REM sleep and non-REM sleep. Normally, sleep is includes a combination of non-REM sleep, which is the rest period of the brain, and REM sleep, which is the rest period of the body. It is known that, in healthy adults, REM sleep and non-REM sleep are repeated several times during the night, followed by awakening in the morning. In favorable sleep, non-REM sleep, which is the rest period of the brain, mainly occurs immediately after falling asleep, and the duration of non-REM sleep is long. It is known that, in contrast, people who complain of insomnia have light sleep, and measurement of their sleep based on brain waves has revealed that the duration of non-REM sleep is shorter in those people than in people with satisfactory sleep.

Techniques for improvement of sleep in such people suffering from insomnia utilize widely known effects of naturally occurring ingredients. Cedrol, which is an aroma ingredient contained in conifers such as Japanese cypress and hiba trees, is known to have an effect of increasing total sleep time, shortening sleep onset latency, and increasing sleep efficiency (see, for example, WO 01/058435 and Pharmacology Biochemistry & Behavior, Vol. 17, pp. 65-71, 1982).

A brain dysfunction-ameliorating agent containing astaxanthin as an active ingredient has been disclosed, and sleep disorder is described as an example of brain dysfunction (see, for example, Japanese Patent Application Laid-Open (JP-A) No. 2007-126455). There is also a description reporting that a composition containing a combination of a carotenoid and a red-wine polyphenol has a sleep-improving effect (see JP-A No. 2009-159929).

### SUMMARY OF INVENTION

### Technical Problem

For improvement of sleep, a feeling of sound sleep is required in addition to increasing the duration of sleep. It has been thought that a feeling of sound sleep can be obtained by sedation during sleep, such as an increased duration of non-REM sleep, a shortened sleep onset latency, and reduced interruption of sleep. At present, there is no composition having a sleep-improving effect which increases the duration of non-REM sleep. For example, the brain dysfunction-ameliorating agent described in JP-A No. 2007-126455 cannot be said to have a sufficient sleep-improving effect, since the literature does not describe that the brain dysfunction-ameliorating agent has an effect of increasing the duration of non-REM sleep. The composition containing a combination of a carotenoid and a red-wine polyphenol described in JP-A No. 2009-159929 also cannot be said to have a sufficient sleep-improving effect, since the literature does not describe that the composition has an effect of increasing the duration of non-REM sleep, although the composition is described to have a sleep-improving effect.

As described above, development of a sleep-improving agent, a non-REM sleep time-increasing agent, and a sedative agent, with which a feeling of sound sleep can be obtained, is in demand.

An object of the invention is to provide a sleep-improving agent, a non-REM sleep time-increasing agent, and a sedative agent, with which a feeling of sound sleep can be obtained.

### Solution to Problem

That is, the invention is as follows.

[1] A sleep-improving agent including, as active ingredients, a lipid-soluble antioxidant and a divalent metal.

[2] The sleep-improving agent according to [1], wherein the lipid-soluble antioxidant is a carotenoid.

[3] The sleep-improving agent according to [1] or [2], wherein the lipid-soluble antioxidant is at least one selected from the group consisting of astaxanthin and derivatives thereof.

[4] The sleep-improving agent according to any one of [1] to [3], wherein the lipid-soluble antioxidant is contained in a powder composition obtained by drying an emulsion composition containing: (a) at least one selected from the group consisting of sucrose fatty acid esters and polyglycerol fatty acid esters; and (b) a phospholipid; wherein (a) and (b) have an equal content ratio by mass, or (a) has a higher content ratio by mass.

[5] The sleep-improving agent according to any one of [1] to [4], wherein the divalent metal is incorporated in a yeast.

[6] The sleep-improving agent according to any one of [1] to [5], wherein the divalent metal is zinc.

[7] The sleep-improving agent according to any one of [1] to [6], wherein the mass ratio of the lipid-soluble antioxidant and the divalent metal is from 1:0.01 to 1:10

[8] The sleep-improving agent according to any one of [1] to [7], further comprising a water-soluble antioxidant.

[9] The sleep-improving agent according to [8], wherein the water-soluble antioxidant is at least one selected from the group consisting of ascorbic acid, derivatives thereof, and thioctic acid.

[10] The sleep-improving agent according to [8] or [9], wherein the mass ratio of the water-soluble antioxidant and the lipid-soluble antioxidant is from 1:0.01 to 1:10.

[11] The sleep-improving agent according to [1], wherein the lipid-soluble antioxidant is astaxanthin, and the divalent metal is zinc.

[12] The sleep-improving agent according to [11], wherein the lipid-soluble antioxidant is astaxanthin, and the lipid-soluble antioxidant is contained in a powder composition obtained by drying an emulsion composition comprising: (a) at least one selected from the group consisting of a sucrose fatty acid ester and a polyglycerol fatty acid ester; and (b) a phospholipid, wherein (a) and (b) have an equal content ratio by mass, or (a) has a higher content ratio by mass.

[13] The sleep-improving agent according to [12], wherein the lipid-soluble antioxidant is astaxanthin, and the astaxanthin is contained in an astaxanthin-containing oil.

[14] A non-REM sleep time-increasing agent including, as active ingredients, a lipid-soluble antioxidant and a divalent metal.

[15] The non-REM sleep time-increasing agent according to [14], wherein the lipid-soluble antioxidant is astaxanthin, and the divalent metal is zinc.

[16] The non-REM sleep time-increasing agent according to[15], wherein the lipid-soluble antioxidant is astaxanthin, and the lipid-soluble antioxidant is contained in a powder composition obtained by drying an emulsion composition comprising: (a) at least one selected from the group consisting of a sucrose fatty acid ester and a polyglycerol fatty acid ester; and (b) a phospholipid, wherein (a) and (b) have an equal content ratio by mass, or (a) has a higher content ratio by mass.

[17] The non-REM sleep time-increasing agent according to [16], wherein the lipid-soluble antioxidant is astaxanthin, and the astaxanthin is contained in an astaxanthin-containing oil.

[18] A sedative including, as active ingredients, a lipid-soluble antioxidant and a divalent metal.

### Advantageous Effects of Invention

The invention can provide a sleep-improving agent, non-REM sleep time-increasing agent, and sedative, with which a feeling of sound sleep can be obtained.

### BRIEF DESCRIPTION OF DRAWINGS

FIG. 1A is a graph showing measurement results of the duration of non-REM sleep in mice after administration of the samples of Example 1 of the invention, Comparative Example 1, and Comparative Example 2.

FIG. 1B is a graph showing measurement results of the duration of awakening in mice after administration of the samples of Example 1 of the invention, Comparative Example 1, and Comparative Example 2.

FIG. 1C is a graph showing measurement results of the duration of REM sleep in mice after administration of the samples of Example 1 of the invention, Comparative Example 1, and Comparative Example 2.

FIG. 2 is a graph showing a comparison of the number of times of actions by mice after administration of the samples of Example 6 of the invention, and Comparative Examples 6 to 8.

FIG. 3 is a graph showing a comparison of the number of times of actions by mice after administration of the samples of Comparative Examples 9 and 10.

### DESCRIPTION OF EMBODIMENTS

Each of the sleep-improving agent, non-REM sleep time-increasing agent, and sedative agent of the invention includes, as active ingredients, a lipid-soluble antioxidant and a divalent metal.

That is, by combination of a lipid-soluble antioxidant and a divalent metal, the invention can achieve an excellent feeling of sound sleep, increase in the duration of non-REM sleep, or a sedating effect, which cannot be achieved by singly using a lipid-soluble antioxidant or a divalent metal.

More specifically, in general, REM sleep and non-REM sleep are repeated during sleep. Non-REM sleep, which is deep and sound sleep, continues mainly during about the first 3 hours of sleep. A sleep disorder occurs due to the absence, or shortening of the deep non-REM sleep, and causes deterioration of the quality of sleep. In the case of insomnia, long sleep onset latency or interruption of sleep may frequently occur, and deterioration of the quality of sleep similarly occurs.

By use of the sleep-improving agent, the non-REM sleep time-increasing agent, or the sedative agent of the invention, the duration of non-REM sleep can be increased; a feeling of sound sleep can be obtained due to shortening of sleep onset latency, reduced interruption of sleep, and favorable awakening; and a favorable feeling at the time of awakening can be obtained, resulting in an improved quality of sleep.

“Improved sleep” in the invention means a high level of a feeling of sound sleep, and includes an increase in both the duration of non-REM sleep and sedation during sleep. In the invention, “sedation” means sedation during sleep, and means short sleep onset latency, reduced interruption of sleep, and favorable awakening.

In the present specification, the term “process” encompasses an independent process, as well as a process that cannot be clearly distinguished from another process but yet achieves the expected effect of the process of interest.

In the present specification, any numerical range expressed herein using “to” refers to a range including the numerical values before and after “to” as the minimum and maximum values, respectively.

In a case in which the amount of an ingredient in the composition is indicated in the invention, when there are plural substances corresponding to the ingredient in the composition, the indicated amount means the total amount of the plural substances present in the composition, unless specifically stated otherwise.

The invention is described below.

The sleep-improving agent of the invention includes, as active ingredients, a lipid-soluble antioxidant and a divalent metal.

The lipid-soluble antioxidant in the invention is preferably an ingredient having a solubility of less than 0.5 g/L in water at 20° C. Specific examples of the lipid-soluble antioxidant include carotenoids, fat-soluble vitamins, fat-soluble vitamin-like substances, and ω-3 oils. In particular, at least one selected from the group consisting of carotenoids and fat-soluble vitamin-like substances is preferred. The lipid-soluble antioxidant is most preferably a carotenoid(s).

Examples of the carotenoid include hydrocarbons (carotenes) and oxidized alcohol derivatives thereof (xanthophylls), and ester derivatives thereof. In the invention, these compounds are also included in the “carotenoid” unless otherwise specified.

Examples of carotenoids which may be preferably used include natural pigments. Examples of the carotenoid which may be applied to the invention include yellow-to-red terpenoid pigments which may be derived from a plant, algae, or bacterium. The carotenoid is not limited to a naturally occurring carotenoid, but also includes a carotenoid obtained by synthesis or biosynthesis. Examples of the carotenoid include actinioerythrol, astaxanthin, bixin, canthaxanthin, capsanthin, capsorubin, β-8′-apo-carotenal (apocarotenal), 62 -12′-apo-carotenal, α-carotene, β-carotene, “carotene” (a mixture of α- and β-carotenes), γ-carotene, β-cryptoxanthin, echinenone, lutein, lycopene, violaxanthin, and zeaxanthin. These carotenoids may be ester derivatives of carotenoids containing a hydroxyl group or carboxyl group.

From the viewpoint of sleep-improving effect, the carotenoid in the invention is preferably astaxanthin, which is known as a yellow-to-red coloring agent. Astaxanthin may be contained in the sleep-improving agent of the invention as a component in an astaxanthin-containing oil separated or extracted from an astaxanthin-containing natural product. Examples of the astaxanthin-containing oil include extracts from cultures obtained by culturing a red yeast Phaffia, green algae Haematococcus, marine bacterium, or the like; and extracts from Antarctic krill, krill powder, shrimp eye powder, or dried salmon powder.

The lipid-soluble antioxidant in the invention may be used in a form of an emulsion composition containing: (a) at least one selected from the group consisting of a sucrose fatty acid ester and a polyglycerol fatty acid ester; and (b) a phospholipid; wherein (a) and (b) have an equal content ratio or (a) has a higher content ratio. The emulsion composition may also be preferably used as a powder composition obtained by drying the emulsion composition. In particular, in a case where a carotenoid such as astaxanthin is used as the lipid-soluble antioxidant, the carotenoid is preferably in the form of this kind of emulsion composition or powder composition in view of increasing the absorbability of the lipid-soluble antioxidant in the body.

(a) At Least One Selected from Group Consisting of Sucrose Fatty Acid Ester and Polyglycerol Fatty Acid Ester

The emulsion composition containing the lipid-soluble antioxidant, or powder composition obtained by drying the emulsion composition in the invention preferably contains at least one selected from the group consisting of a sucrose fatty acid ester and a polyglycerol fatty acid ester.

Both the sucrose fatty acid ester and the polyglycerol fatty acid ester functions as surfactants, and can reduce the average particle diameter of the emulsion particles in the emulsion composition.

From the viewpoint of surfactant performance, the sucrose fatty acid ester that may be used in the invention is preferably a sucrose fatty acid ester having a fatty acid of 12 or more carbon atoms, and more preferably a sucrose fatty acid ester having a fatty acid of 12 to 20 carbon atoms. In a case in which the sucrose fatty acid ester has a fatty acid of 12 or more carbon atoms, emulsion particles having a smaller average particle diameter may be produced in some cases.

Examples of the sucrose fatty acid ester include sucrose dioleic acid ester, sucrose distearic acid ester, sucrose dipalmitic acid ester, sucrose dimyristic acid ester, sucrose dilauric acid ester, sucrose monooleic acid ester, sucrose monostearic acid ester, sucrose monopalmitic acid ester, sucrose monomyristic acid ester, and sucrose monolauric acid ester. Among these sucrose fatty acid esters, sucrose monooleic acid ester, sucrose monostearic acid ester, sucrose monopalmitic acid ester, sucrose monomyristic acid ester, and sucrose monolauric acid ester are preferred, and sucrose monolauric acid ester and sucrose monooleic acid ester are more preferred.

In the invention, these sucrose fatty acid esters may be used singly, or in mixture of two or more kinds thereof.

As the sucrose fatty acid ester, a commercially available product may be used. Examples of the commercially available product include RYOTO Sugar Esters S-070, S-170, S-270, S-370, S-370F, S-570, S-770, S-970, S-1170, S-1170F, S-1570, S-1670, P-070, P-170, P-1570, P-1670, M-1695, 0-170, 0-1570, OWA-1570, L-195, L-595, L-1695, LWA-1570, B-370, B-370F, ER-190, ER-290, and POS-135, manufactured by Mitsubishi-Kagaku Foods Corporation; and DK Esters SS, F160, F140, F110, F90, F70, F50, F-A50, F-20W, F-10, and F-A10E, and Cosmelike B-30, S-10, S-50, S-70, S-110, S-160, S-190, SA-10, SA-50, P-10, P-160, M-160, L-10, L-50, L-160, L-150A, L-160A, R-10, R-20, 0-10, and 0-150, manufactured by Dai-ichi Kogyo Seiyaku Co., Ltd.

Examples of polyglycerol fatty acid esters which may be used in the invention include esters of formed between a polyglycerol having an average degree of polymerization of not less than 2, preferably from 6 to 15, more preferably from 8 to 10, and a fatty acid having from 8 to 18 carbon atoms such as caprylic acid, capric acid, lauric acid, myristic acid, palmitic acid, stearic acid, oleic acid, or linoleic acid.

Examples of the polyglycerol fatty acid esters include glyceryl hexaglycerol monooleate, hexaglycerol monostearic acid ester, hexane glycerol monopalmitic acid ester, hexaglycerol monomyristic acid ester, hexaglycerol monolauric acid ester, decaglycerol monooleic acid ester, decaglycerol monostearic acid ester, decaglycerol monopalmitic acid ester, decaglycerol monomyristic acid ester, and decaglycerol monolauric acid ester. Among these polyglycerol fatty acid esters, decaglycerol monooleic acid ester (HLB=12), decaglycerol monostearic acid ester (HLB=12), decaglycerol monopalmitic acid ester (HLB=13), decaglycerol monomyristic acid ester (HLB=14), and decaglycerol monolauric acid ester (HLB=16) are preferred.

These polyglycerol fatty acid esters may be used singly, or in mixture of two or more kinds thereof.

As the polyglycerol fatty acid ester, a commercially available product may be used. Examples of the commercially available product include NIKKOL DGMS, NIKKOL DGMO-CV, NIKKOL DGMO-90V, NIKKOL DGDO, NIKKOL DGMIS, NIKKOL DGTIS, NIKKOL Tetraglyn 1-SV, NIKKOL Tetraglyn 1-O, NIKKOL Tetraglyn 3-S, NIKKOL Tetraglyn 5-S, NIKKOL Tetraglyn 5-O, NIKKOL Hexaglyn 1-L, NIKKOL Hexaglyn 1-M, NIKKOL Hexaglyn 1-SV, NIKKOL Hexaglyn 1-O, NIKKOL Hexaglyn 3-S, NIKKOL Hexaglyn 4-B, NIKKOL Hexaglyn 5-S, NIKKOL Hexaglyn 5-O, NIKKOL Hexaglyn PR-15, NIKKOL Decaglyn 1-L, NIKKOL Decaglyn 1-M, NIKKOL Decaglyn 1-SV, NIKKOL Decaglyn 1-50SV, NIKKOL Decaglyn 1-ISV, NIKKOL Decaglyn 1-O, NIKKOL Decaglyn 1-OV, NIKKOL Decaglyn 1-LN, NIKKOL Decaglyn 2-SV, NIKKOL Decaglyn 2-ISV, NIKKOL Decaglyn 3-SV, NIKKOL Decaglyn 3-OV, NIKKOL Decaglyn 5-SV, NIKKOL Decaglyn 5-HS, NIKKOL Decaglyn 5-IS, NIKKOL Decaglyn 5-OV, NIKKOL Decaglyn 5-0-R, NIKKOL Decaglyn 7-S, NIKKOL Decaglyn 7-O, NIKKOL Decaglyn 10-SV, NIKKOL Decaglyn 10-IS, NIKKOL Decaglyn 10-OV, NIKKOL Decaglyn 10-MAC, and NIKKOL Decaglyn PR-20, manufactured by Nikko Chemicals Co., Ltd.; Ryoto Polyglyester L-7D, L-10D, M-10D, P-8D, SWA-10D, SWA-15D, SWA-20D, S-24D, S-28D, O-15D, O-50D, B-70D, B-100D, ER-60D, LOP-120DP, DS13W, DS3, HS11, HS9, TS4, TS2, DL15, and DO13, manufactured by Mitsubishi-Kagaku Foods Corporation; SUNSOFT Q-17UL, SUNSOFT Q-14S, and SUNSOFT A-141C, manufactured by Taiyo Chemicals Co., Ltd.; and POEM DO-100 and POEM J-0021, manufactured by Riken Vitamin Co., Ltd.

The at least one ingredient selected from the group consisting of the sucrose fatty acid ester and the polyglycerol fatty acid ester is contained at preferably from 1% by mass to 50% by mass, more preferably from 1% by mass to 30% by mass, and still more preferably from 1% by mass to 10% by mass with respect to the total mass of the emulsion composition containing the lipid-soluble antioxidant such as astaxanthin, from the viewpoint of emulsion stability and storage stability after re-dissolution.

The emulsion composition containing the lipid-soluble antioxidant such as astaxanthin contains at least one of a sucrose fatty acid ester or a polyglycerol fatty acid ester selected from these sucrose fatty acid esters or polyglycerol fatty acid esters. From the viewpoint of increasing the storage stability of the composition in the form of a powder, the sucrose fatty acid ester and polyglycerol fatty acid ester are preferably used in combination. In cases where a sucrose fatty acid ester(s) is/are used in combination with a polyglycerol fatty acid ester(s), the mass ratio between the sucrose fatty acid ester(s) and polyglycerol fatty acid ester(s) is preferably from 1:9 to 9:1, and more preferably from 2:8 to 8:2 from the viewpoint of increasing the storage stability of the composition in the form of a powder, although the mass ratio is not limited.

The sucrose fatty acid ester(s) and polyglycerol fatty acid ester(s) have HLB values of preferably 8 or more, more preferably 10 or more, still more preferably 12 or more. Although there is no upper limit of the HLB values, the HLB values are generally 18 or less, preferably 17 or less.

The HLB values can be determined according to a calculation equation generally used in the field of ordinary surfactants for determining the hydrophilicity-hydrophobicity balance, such as the Kawakami's equation. The invention uses the following Kawakami's equation.

HLB=7+11.7 log(Mw/Mo)

Mw herein represents the molecular weight of hydrophilic groups, and Mo represents the molecular weight of hydrophobic groups.

HLB values described in catalogs and the like may be used.

(b) Phospholipid

The emulsion composition containing the lipid-soluble antioxidant or the powder composition obtained by drying the emulsion composition in the invention preferably contains a phospholipid.

Examples of the phospholipid which may be used in the invention include glycerophospholipids containing no glycerol, and sphingophospholipids containing sphingoid bases. Glycerophospholipids are preferred.

Examples of glycerophospholipids which may be used in the invention include ingredients such as phosphatidic acid, bisphosphatidic acid, lecithin (phosphatidyl choline), phosphatidyl ethanolamine, phosphatidylmethylethanolamine, phosphatidylserine, phosphatidylinositol, phosphatidylglycerin, and diphosphatidylglycerin (cardiolipin). Examples of the glycerophospholipids also include plant-derived glycerophospholipids such as glycerophospholipids derived from soybean, maize, peanut, rapeseed, or wheat containing these ingredients; animal-derived glycerophospholipids such as glycerophospholipids derived from yolk or bovine containing these ingredients; and lecithins derived from microorganisms such as E. coli.

In the invention, examples of the glycerophospholipids also include glycerophospholipids having a single fatty acid residue per molecule as a result of enzymatic degradation, that is, lysolecithin. Such lysolecithin can be obtained by hydrolysis of lecithin by an acid or alkali catalyst, or can be obtained by hydrolysis of lecithin by phospholipase A1 or A2.

Examples of the lysolecithin include lysophosphatidic acid, lysophosphatidylglycerol, lysophosphatidylinositol, lysophosphatidylethanolamine, lysophosphatidylmethylethanolamine, lysophosphatidylcholine (lysolecithin), and lysophosphatidylserine.

The glycerophospholipids represented by lecithin may be hydrogenated or hydroxylated for use in the invention.

The hydrogenation is carried out by, for example, reacting lecithin with hydrogen in the presence of a catalyst. The hydrogenation occurs at an unsaturated bond of a fatty acid moiety. The hydrogenation improves the oxidation stability of the lecithin.

The hydroxylation is carried out by heating lecithin together with high concentrations of hydrogen peroxide and an organic acid such as acetic acid, tartaric acid, or butyric acid. The hydroxylation occurs at an unsaturated bond of a fatty acid moiety. The hydroxylation improves the hydrophilicity of the lecithin.

The phospholipid preferably has two fatty acid residues per molecule from the viewpoint of storage stability of the astaxanthin-containing emulsion composition in the form of a powder. More specifically, lecithin is preferred.

Since lecithin has a hydrophilic group and hydrophobic group in the molecule, lecithin is widely used as emulsifiers in the fields of food, pharmaceuticals and cosmetics.

The content of the phospholipid is preferably from 0.1% by mass to 5% by mass, and more preferably from 0.2% by mass to 3% by mass with respect to a total mass of the emulsion composition containing the lipid-soluble antioxidant such as astaxanthin, from the viewpoint of emulsion stability and storage stability after re-dissolution.

In a case where the content of the phospholipid is 0.1% by mass or more, the stability of the emulsion composition containing the lipid-soluble antioxidant such as astaxanthin tends to be high.

The composition ratio by mass of the at least one ingredient selected from the group consisting of the sucrose fatty acid ester and the polyglycerol fatty acid ester, and the phospholipid, contained in the emulsion composition containing the lipid-soluble antioxidant such as astaxanthin in the invention is preferably from 1:1 to 100:1, more preferably from 2:1 to 50:1, and still more preferably 3:1 to 10:1, from the viewpoint of an amount suitable for refinement and emulsion stability of the emulsion composition.

The amount of the lipid-soluble antioxidant to be used for the sleep-improving agent of the invention is not limited as long as the sleep-improving effect can be exhibited. More specifically, the amount of the lipid-soluble antioxidant used is preferably from 1 mg to 1000 mg, more preferably from 2 mg to 300 mg, and still more preferably from 3 mg to 100 mg, per day.

The divalent metal in the invention means a metal which may have a valence of 2. Specific examples of the divalent metal include calcium, magnesium, iron, zinc, selenium, chromium, manganese, copper, and molybdenum. The divalent metal is preferably at least one selected from the group consisting of calcium, magnesium, iron, zinc, selenium, and chromium, more preferably at least one selected from the group consisting of calcium, magnesium, selenium, and zinc. Zinc is especially preferred since zinc, when combined with a lipid-soluble antioxidant, has an effect of remarkably increasing the amount of non-REM sleep.

The amount of the divalent metal to be used is not limited as long as the sleep-improving effect can be exhibited. More specifically, the amount of the divalent metal is preferably from 1 mg to 300 mg, more preferably from 2 mg to 100 mg, and still more preferably from 3 mg to 50 mg, per day.

The divalent metal may be present in the composition in the form of a single substance, in a form in which the divalent metal is bound to a protein or the like, in the form of an ion, or in a form in which the divalent metal is incorporated in a yeast (mineral yeast). The divalent metal is preferably in a form in which the divalent metal is incorporated in the yeast (mineral yeast). The divalent metal may also be used in the form of a salt such as a gluconic acid salt (for example, zinc gluconate)

A mineral yeast means yeast containing a mineral absorbed in the cell, which is prepared by culturing yeast in a culture medium supplemented with a high concentration of the mineral (for example, calcium, magnesium, iron, zinc, selenium, chromium, manganese, copper, or molybdenum). The mineral yeast can be obtained by culturing a yeast in a medium supplemented with a mineral and then collecting the cells, followed by concentration, sterilization, drying, and/or the like. The mineral yeast may be a commercially available mineral yeast. Examples of yeasts that may be used include yeasts belonging to the genus Saccharomyces, genus Mycotorula, and genus Torulopsis; food yeasts such as baker's yeast, beer yeast, wine yeast, sake yeast, alcohol yeast, and miso/shoyu yeast; and various other types of yeasts.

Since the mineral is incorporated in the cell of such mineral yeast, the mineral yeast can be ingested without feeling a metallic taste. Moreover, since the mineral is incorporated in the cell of the mineral yeast, the mineral is bound to a protein so as to be present in the form of an organic substance. Thus, application of mineral yeast to a mammal such as a human allows better absorption of the mineral in the body compared to inorganic minerals.

In cases where a divalent metal is used in a form in which the divalent metal is incorporated in the yeast, the amount of the divalent metal used per day is preferably from 5 mg to 6000 mg, more preferably from 20 mg to 2000 mg, and still more preferably from 25 mg to 300 mg, in terms of the mass of the yeast containing the divalent metal incorporated therein.

In the invention, the mass ratio of the lipid-soluble antioxidant and the divalent metal is preferably from 1:0.01 to 1:10, and more preferably from 1:0.1 to 1:5, from the viewpoint of the sleep-improving effect. The mass of the divalent metal in this case means the mass as the amount of the divalent metal, irrespective of the form of the divalent metal.

The sleep-improving agent of the invention may further contain a water-soluble antioxidant. By inclusion of the water-soluble antioxidant, the stability of the lipid-soluble antioxidant increases, and a higher sleep-improving effect can be expected as a result.

The water-soluble antioxidant in the invention is preferably an ingredient having a solubility of not less than 0.5 g/L in water at 20° C. More specifically, the water-soluble antioxidant is preferably at least one selected from the group consisting of ascorbic acid and derivatives thereof, thioctic acid, catechins, and flavonoids. The water-soluble antioxidant is more preferably at least one selected from the group consisting of ascorbic acid and derivatives thereof, and thioctic acid.

The ascorbic acid and derivatives thereof are not limited, and examples of the ascorbic acid and derivatives thereof include synthesized products which are commonly used and extracts derived from natural ingredients.

The ascorbic acid and derivatives thereof are preferably water-soluble ascorbic acid and derivatives thereof.

Specific examples of the ascorbic acid and ascorbic acid derivatives include ascorbic acid, sodium ascorbate, potassium ascorbate, calcium ascorbate, L-ascorbic acid phosphate, magnesium ascorbyl phosphate, ascorbyl sulfate, disodium ascorbyl sulfate, and ascorbyl-2-glucoside. Other examples of the ascorbic acid and derivatives thereof in the invention include erythorbic acid and derivatives thereof such as erythorbic acid, sodium erythorbate, potassium erythorbate, calcium erythorbate, erythorbic acid phosphate, and erythorbic acid sulfate.

These ascorbic acid and derivatives thereof may be ascorbic acid and derivatives thereof which are commercially available. Examples of the ascorbic acid and derivatives thereof which are commercially available include L-ascorbic acid (Takeda Pharmaceutical Company Limited, Fuso Chemical Co., Ltd., BASF Japan Ltd., Daiichi Pharmaceutical Co., Ltd., and the like), L-ascorbic acid Na (Takeda Pharmaceutical Company Limited, Fuso Chemical Co., Ltd., BASF Japan Ltd., Daiichi Pharmaceutical Co., Ltd., and the like), ascorbic acid-2-glucoside (trade name, AA-2G; Hayashibara Biochemical Laboratories Inc.), and L-ascorbic acid phosphate Mg (trade name, ascorbic acid PM “SDK” (Showa Denko K. K.); trade name, NIKKOL VC-PMG (Nikko Chemicals Co., Ltd.); trade name, C-MATE (Takeda Pharmaceutical Company Limited)).

The amount of the ascorbic acid and/or derivative(s) thereof to be used is not limited as long as the sleep-improving effect of the active ingredients can be increased. More specifically, the amount of the ascorbic acid and/or derivative(s) thereof to be used may be from 5 mg to 2000 mg, and preferably from 30 mg to 500 mg, per day.

The thioctic acid in the invention is also called α-lipoic acid, and examples of the thioctic acid include, but are not limited to, synthesized products which are commonly used, and extracts derived from natural ingredients.

The thioctic acid may be used as it is as a powder, but is preferably used together with an emulsifier so that the powder can easily disperse in an aqueous solution. Examples of the method of dispersion using an emulsifier which may be used include the method described in JP-A No. 2007-16000.

The thioctic acid is preferably used as a cyclodextrin clathrate. Such use prevents reaction caused by contacting of the thioctic acid with other antioxidants, and improves the temporal stability. Examples of the method of clathrating the thioctic acid in cyclodextrin include common methods such as the method described in JP-A No. 2006-169253.

The amount of the thioctic acid to be used is not limited as long as the sleep-improving effect of the active ingredients can be increased. More specifically, the amount of the thioctic acid is preferably from 1 mg to 1000 mg, and more preferably from 3 mg to 200 mg, per day.

In the invention, the mass ratio of the water-soluble antioxidant and the lipid-soluble antioxidant is preferably from 1:0.01 to 1:10, and more preferably from 1:0.1 to 1:5, from the viewpoint of the sleep-improving effect of the active ingredients.

The sleep-improving agent of the invention is preferably applied to foods and pharmaceuticals. Examples of the foods include, but are not limited to, beverages (including powdered drinks and alcoholic beverages); frozen desserts; and processed foods such as rice balls, sandwiches, soups, instant noodles, and rice gruels. Examples of the pharmaceuticals include, but are not limited to, energy drinks and analeptics.

To the sleep-improving agent of the invention, arbitrary ingredients which can be added to foods and pharmaceuticals may be further added.

In cases where the agent is prepared as a solution, examples of carriers which may be preferably used include aqueous media such as water. In cases where the agent is prepared as a solid, examples of additive ingredients which may be preferably used include vehicles such as crystalline cellulose and magnesium stearate, and disintegrators such as corn starch and alginic acid.

Other examples of the arbitrary ingredients which can be added to foods and pharmaceuticals include low-hygroscopic materials and moisture absorbents. Examples of low-hygroscopic materials which may be preferably used include celluloses, powdered celluloses, microcrystalline celluloses, lactose, oligosaccharides, sugar alcohols, trehalose, and calcium stearate. Examples of moisture absorbents which may be used include silicates, magnesium carbonate, ferrocyanides, and polysaccharides. More preferred examples of the low-hygroscopic materials include crystalline celluloses, microcrystalline celluloses, and lactose. Examples of compounds required for preparing the agent into the form of a powder, solid, or liquid include erythritol, maltitol, hydroxypropylcellulose, kaolin, and talc.

The dosage form of the sleep-improving agent of the invention is not limited, and the agent may be administered either orally or parenterally. Examples of the formulation for oral administration include solid dosage forms such as tablets, orally rapidly disintegrating tablets, capsules, granules, and fine granules; and liquid dosage forms such as syrups and suspensions. Examples of the formulation for parenteral administration include injection solutions, eye drops, patches, ointments, and suppositories. The dosage form of the sleep-improving agent of the invention is preferably oral dosing, and solid dosage forms with capsule formulations are preferred in view of ease of dosing.

In a case where the sleep-improving agent of the invention is used as a capsule formulation, the capsule formulation may be in the form of a hard capsule, soft capsule, microcapsule, seamless capsule, or the like. These capsule formulations preferably have a capsule coating containing one or more of pig skin gelatin, pig bone gelatin, fish gelatin, or natural hydrophilic polymer. These capsule coatings can be prepared by well-known ordinary methods. The term “capsule coating containing pig skin gelatin, pig bone gelatin, fish gelatin, and/or natural hydrophilic polymer” means that the total amount of pig skin gelatin, pig bone gelatin, fish gelatin, and/or natural hydrophilic polymer is 30% by mass or more, preferably 40% by mass or more, more preferably 50% by mass or more, especially preferably 60% mass or more, with respect to the total mass of the capsule coating. As long as the effect of the invention is not deteriorated, other materials such as bovine skin gelatin may be contained in the capsule coating.

The natural hydrophilic polymer is a hydrophilic polymer derived from a natural animal, plant, or the like, or is a processed polymer thereof, which polymer is obtained by purification of the material or by synthesis using the material. Examples of the natural hydrophilic polymer include at least one selected from the group consisting of alginic acid and salts thereof, agar gum, guar gum, carob bean gum, tara gum, gum ghatti, Khaya grandifolia gum, gum tragacanth, karaya gum, pectin, gum arabic, xanthan gum, gellan gum, starch, konjac mannan, galactomannan, funoran, acetan gum, welan, rhamsan, furcellaran, succinoglycan, scleroglycan, schizophyllan, tamarind gum, curdlan, carrageenan, pullulan, and dextran. These natural hydrophilic polymers may be used in combination of two or more kinds thereof, or the natural hydrophilic polymer(s) may be used in combination with the pig skin gelatin and/or the like described above. These natural hydrophilic polymers may be polymers obtained by processing natural products. The natural hydrophilic polymer is especially preferably at least one selected from the group consisting of pullulan, carrageenan, and dextran. Carrageenan is especially preferred.

The pig skin gelatin, pig bone gelatin, and fish gelatin mean proteins prepared by warm-water extraction of proteins obtained using, as a material, pig skin, pig bone, or fish, respectively. The pig skin gelatin, pig bone gelatin, and fish gelatin in the invention can be obtained by, for example, treating pig skin, pig bone, or fish such as Perciformes, cod, tuna, deep-sea fish, or the like with an acid or alkali, and then warming the treated product in water to perform extraction to obtain an extract, followed by purifying the obtained extract through an ion-exchange treatment process.

The pig skin gelatin, pig bone gelatin, fish gelatin, or natural hydrophilic polymer can be converted into small molecules by enzymatic treatment or the like. The average molecular weight of the pig skin gelatin, pig bone gelatin, fish gelatin, or natural hydrophilic polymer may be selected, if appropriate, and is usually from about 10,000 to about 5,000,000, preferably from about 10,000 to about 5,000,000, more preferably from about 10,000 to about 2,500,000, still more preferably from about 10,000 to about 1,000,000, and particularly preferably from about 10,000 to about 500,000.

The capsule coating to be used for the capsule formulation may contain not only the material derived from a specific animal, plant, or the like described above, but also one or more of oils, polyols, surfactants, antioxidants, pigments, or flavoring agents. Examples of the oils include natural oils such as evening primrose oil, soybean oil, safflower oil, olive oil, germ oil, rapeseed oil, sunflower oil, peanut oil, cottonseed oil, rice bran oil, and coca butter, and hydrogenated oils thereof; and glycerides (glycerides, diglycerides, triglycerides, and the like) of fatty acids. Examples of the polyols include polyethylene glycol, propylene glycol, glycerin, and sorbitol. Examples of the surfactants include nonionic surfactants such as sorbitan fatty acid esters and polyglycerol fatty acid esters. Examples of the pigments include carotenoid pigments, anthocyanin pigments, cacao pigments, anthraquinone pigments, and caramel pigments. In particular, addition of one or more of oils, polyalcohols, surfactants, and/or natural pigments to the capsule coating is preferred from the viewpoint of increasing the stability of the capsule formulation.

The sleep-improving agent of the invention can be in the form of the formulation described above which contains effective amounts of ingredients. For example, the sleep-improving agent may contain, as a dosage form for once-daily administration, from 1 mg to 1000 mg of the lipid-soluble antioxidant and from 1 mg to 300 mg of the divalent metal.

The sleep-improving agent of the invention may also be a sleep-improving agent which contains from 1 mg to 1000 mg of the lipid-soluble antioxidant and from 1 mg to 300 mg of the divalent metal, and in which the mass ratio of the lipid-soluble antioxidant and the divalent metal is from 1:0.01 to 1:10.

By taking the sleep-improving agent of the invention, a favorable feeling of sound sleep can be obtained. The sleep-improving agent is preferably taken at bedtime, more preferably taken from 0.5 hour to 6 hours before bedtime, still more preferably taken from 1 hour to 3 hours before bedtime.

The dose of the sleep-improving agent of the invention is from about 0.001 mg/kg/day to about 10,000 mg/kg/day, preferably from about 2.5 mg/kg/day to about 20 mg/kg/day, although the dose may vary depending on the age, body weight, dosing method, and the like of the taker.

The non-REM sleep time-increasing agent of the invention contains, as active ingredients, a lipid-soluble antioxidant and a divalent metal. By taking the non-REM sleep time-increasing agent of the invention, the amount of non-REM sleep can be increased. The non-REM sleep time-increasing agent is preferably taken at bedtime, more preferably taken from 0.5 hour to 6 hours before bedtime, still more preferably taken from 1 hour to 3 hours before bedtime.

The dose of the non-REM sleep time-increasing agent of the invention is from about 0.001 mg/kg/day to about 10,000 mg/kg/day, and preferably from about 2.5 mg/kg/day to about 20 mg/kg/day, although the dose may vary depending on the age, body weight, dosing method, and the like of the taker.

Other matters concerning the non-REM sleep time-increasing agent of the invention are the same as the matters described for the sleep-improving agent of the invention.

The sedative agent of the invention is a sedative agent which is effective during sleep, and contains a lipid-soluble antioxidant and a divalent metal as active ingredients. The sedative of the invention can provide mental sedation during sleep in mammals including human, and reduce the state of tension or stress, providing a relaxing effect.

By taking the sedative agent of the invention at bedtime, one can smoothly fall asleep due to the relaxing effect, and effects such as shortening of sleep onset latency, reduced interruption of sleep, and favorable awakening can be obtained. Thus, the sedative agent is preferably taken at bedtime. The sedative agent is more preferably taken from 0.5 hour to 6 hours before bedtime, still more preferably taken from 1 hour to 3 hours before bedtime.

The dose of the sedative agent of the invention is from about 0.001 mg/kg/day to about 10,000 mg/kg/day, and preferably from about 2.5 mg/kg/day to about 20 mg/kg/day, although the dose may vary depending on the age, body weight, dosing method, and the like of the taker.

Other matters concerning the sedative of the invention are the same as the matters described for the sleep-improving agent of the invention.

### EXAMPLES

The invention is more specifically described below by way of Examples and Comparative Examples. However, the invention is not limited to the Examples.

### Example 1, Comparative Examples 1 and 2

### 1. Methods

### (i) Animals Used

C57BL/6 mice (male; 12 weeks old; body weight: 24-27 g) were purchased from SLC.

(ii) Method of Keeping Mice

The mice were individually maintained at a constant temperature (22±2° C.) and humidity (50±2%) in acrylic cages placed in a sound-proof chamber. The mice were kept under a light/dark cycle of 12/12 hours, and fed with solid feed for mice (feed name: Labo MR Stock). The mice were provided with the food and water ad libitum.

(iii) Operation for Placement of Electrodes for Measuring Brain Waves and Myogenic Potential, and Connection of Electrodes to Measuring Apparatus

The mice were subjected to an operation for placement of electrodes for measuring brain waves and the myogenic potential, and then allowed to recover in a recovery chamber for 10 days. Subsequently, the mice were transferred to a record chamber, and measurement cables were connected to the electrodes, followed by allowing acclimatization of the mice for 4 days.

(iv) Preparation and Administration of Samples

A composition containing the materials described in Table 1 was prepared as an Example sample (Example 1), and orally administered to mice at a dose of 10 g/kg using a sonde needle. The administration was carried out by administering the Example sample to the mice at 16:00 (the time of the beginning of the dark period) (n=7-8). A composition described in Table 1 was used in Comparative Example 1, and water was used in Comparative Example 2, to provide the respective Comparative Example samples. In each Comparative Example, the sample was orally administered to mice at a dose of 10 g/kg. Each value in Table 1 is expressed in milligrams. The percentage of zinc in the beer yeast is expressed in percent by mass.

(v) Recording and Analysis of Brain Waves and Myogenic Potential

The brain waves and myogenic potential were recorded after amplification (brain waves: 0.5-30 Hz; myogenic potential: 20-200 Hz) and subsequent digitization at a sampling rate of 128 Hz. The analysis was carried out using brain-wave recording software “SleepSign” (manufactured by Kissei Comtec Co., Ltd.). Data obtained during 10 seconds were defined as 1 epoch, and each epoch was automatically judged as awakening, non-REM sleep, or REM sleep based on the frequency components and waveforms of the brain waves and myogenic potential. The brain wave data obtained over 4 hours after administration were analyzed to calculate the durations of awakening, non-REM sleep, and REM sleep per hour. In addition, the length of time required for occurrence of non-REM sleep was measured.

TABLE 1

| | | Comparative | Comparative |
|---|---|---|---|
| | Example 1 | Example 1 | Example 2 |
| Astaxanthin | 1.25 | — | — |
| Zinc yeast | 20 | 20 | — |
| (beer yeast containing 10% zinc) | | | |
| Purified water | 250 | 250 | 250 |

2. Results

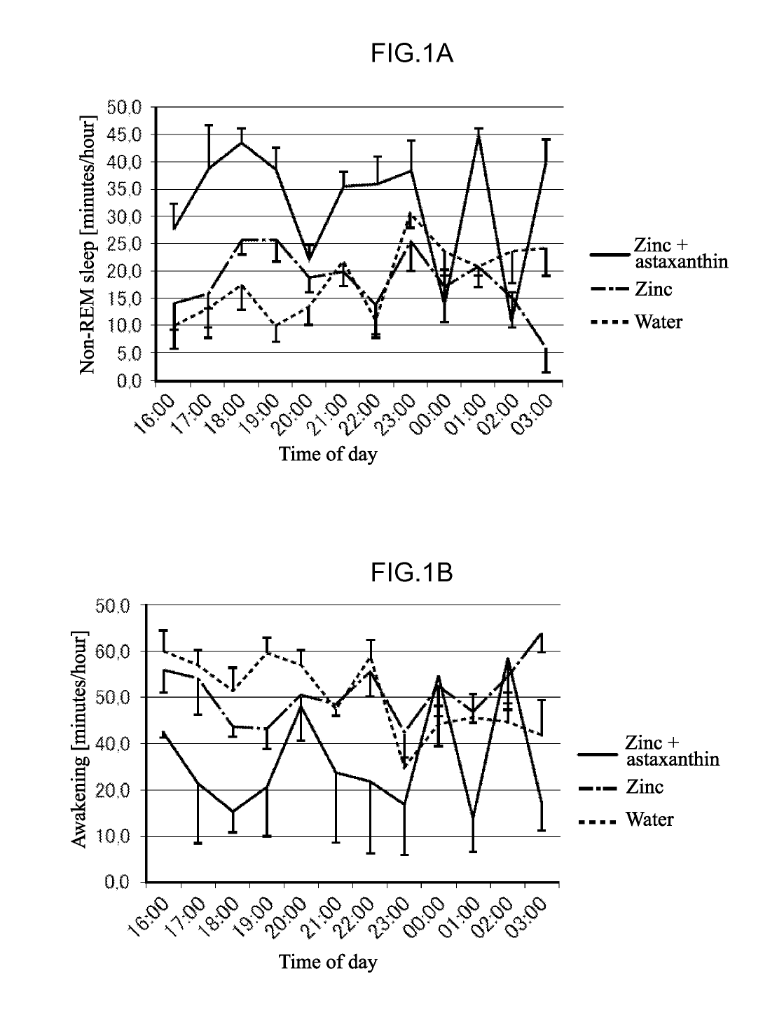

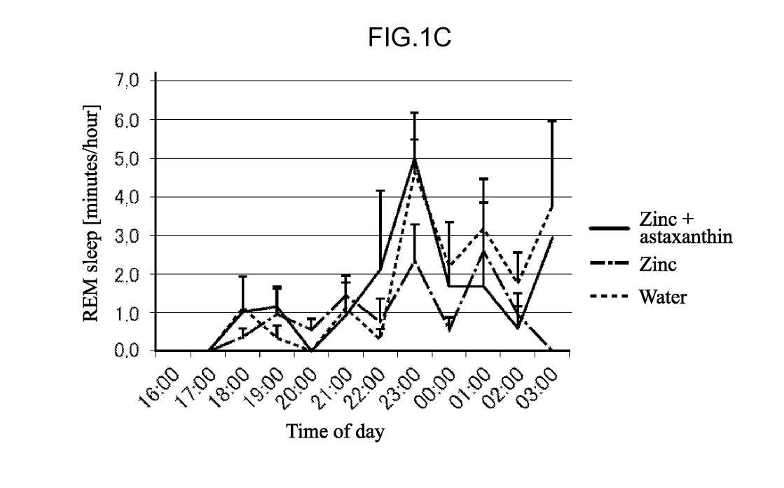

The durations of non-REM sleep, REM sleep, and awakening over 4 hours after administration are shown in Table 2, and the durations of non-REM sleep, REM sleep, and awakening over 12 hours after administration are shown in FIG. 1A to FIG. 1C, respectively.

In Example 1, an effect to significantly increase the duration of non-REM sleep relative to Comparative Examples 1 and 2 could be found at the dose of 10 g/kg. In addition, in Example 1, shortening of the length of time before falling asleep could be found.

TABLE 2

| | | | Length of time |
|---|---|---|---|
| | Duration of | Duration of | before occurrence |
| | non-REM sleep | awakening | of non-REM sleep |
| Example 1 | 119 minutes | 95 minutes | 10 minutes |
| Comparative | 40 minutes | 139 minutes | 70 minutes |
| Example 1 | | | |
| Comparative | 68 minutes | 111 minutes | 70 minutes |
| Example 2 | | | |

### Examples 2 to 4, Comparative Examples 3 and 4

### 1. Methods

### (i) Animals Used

Sprague-Dawley rats (male; 8 weeks old; body weight: 250-280 g) were purchased from SLC.

### (ii) Method of Keeping Rats

The rats were individually maintained in acrylic cages placed in a sound-proof chamber. The rats were kept under a light/dark cycle of 12/12 hours (the time of the beginning of the dark period, 8:00 AM), and fed with solid feed for rats (feed name: Labo MR Stock) . The rats were provided with the food and water ad libitum.

(iii) Operation for Placement of Electrodes for Measuring Brain Waves and Myogenic Potential, and Connection of Electrodes to Measuring Apparatus

The rats were subjected to an operation for placement of electrodes for measuring brain waves and the myogenic potential, and then allowed to recover in a recovery chamber for 10 days. Subsequently, the rats were transferred to a record chamber, and measurement cables were connected to the electrodes, followed by allowing acclimatization of the rats for 4 days.

(iv) Preparation and Administration of Samples

Each of compositions 1 to 3 containing the materials described in Table 3 was prepared as an Example sample or Comparative Example sample. Each value in Table 3 is expressed in milligrams, and the percentage of zinc in the beer yeast is expressed in percent by mass. In Examples 2 to 4, a composition shown in Table 4 (composition 1 or 2) was orally administered to rats at a dose of 6 g/kg or 3 g/kg using a sonde needle. The administration was carried out by administering the Example sample or Comparative Example sample to the rats at 20:00 (the time of the beginning of the dark period) (n=6-7). Water was used in Comparative Example 3, and the composition 3 was used in Comparative Example 4, for oral administration to the rats at a dose of 3 g/kg.

Recording and Analysis of Brain Waves and Myogenic Potential

The brain waves and myogenic potential were recorded after amplification (brain waves, 0.5-30 Hz; myogenic potential, 20-200 Hz) and subsequent digitization at a sampling rate of 128 Hz. The analysis was carried out using brain-wave recording software “SleepSign” (manufactured by Kissei Comtec Co., Ltd.). Data obtained during 10 seconds were defined as 1 epoch, and each epoch was automatically judged as awakening, non-REM sleep, or REM sleep based on the frequency components and waveforms of the brain waves and myogenic potential. The brain wave data obtained over 4 hours after administration were analyzed to calculate the durations of awakening, non-REM sleep, and REM sleep per hour. In addition, the length of time required for occurrence of non-REM sleep was measured.

TABLE 3

| | Composition 1 | Composition 2 | Composition 3 |
|---|---|---|---|
| Astaxanthin | 1.5 | 1.5 | 0 |
| Zinc yeast | 25 | 25 | 25 |
| (beer yeast containing | | | |
| 10% zinc) | | | |
| Ascorbic acid | 25 | 0 | 25 |
| Thioctic acid | 5 | 0 | 5 |

2. Results

The duration of non-REM sleep over 4 hours after administration is shown in Table 4.

In Examples 2 to 4, an effect to significantly increase the duration of non-REM sleep relative to the Comparative Examples could be found both at the dose of 6 g/kg and at the dose of 3 g/kg. In addition, in Examples 2 to 4, shortening of the length of time before falling asleep could be found.

TABLE 4

| | | | | | Length of time |
|---|---|---|---|---|---|
| | | | | | before |
| | | | Duration of | | occurrence of |
| | | Duration of | non-REM | Duration of | non-REM |
| | Sample used | REM sleep | sleep | awakening | sleep |
| Example 2 | Composition 1 | 30 minutes | 100 minutes | 110 minutes | 10 minutes |
| | (6 g) | | | | |
| Example 3 | Composition 1 | 50 minutes | 60 minutes | 130 minutes | 15 minutes |
| | (3 g) | | | | |
| Comparative | Purified water | 70 minutes | 30 minutes | 140 minutes | 70 minutes |
| Example 3 | | | | | |
| Example 4 | Composition 2 | 65 minutes | 45 minutes | 130 minutes | 25 minutes |
| | (3 g) | | | | |
| Comparative | Composition 3 | 65 minutes | 35 minutes | 140 minutes | 55 minutes |
| Example 4 | (3 g) | | | | |

### Example 5, Comparative Example 5

### 1. Methods

### (i) Human Test

Three healthy adult males.

### (ii) Preparation and Administration of Samples

The composition 4 described below or a Comparative Example material (crystalline cellulose, 230 mg) was filled into gelatin capsules, to prepare an Example sample and a Comparative Example sample (Example 5 and Comparative Example 5). Four capsules of one of the Example sample or Comparative Example sample was orally administered, together with 100 ml of water, to every subject 30 minutes before bedtime. Sample administration tests were carried out as follows: first, a placebo sample was continuously orally administered from Monday for 1 week, and, after an interval of one week, the Example sample or Comparative Example sample was orally administered for 1 week. For each sample administration test, symptoms during the sample administration were evaluated based on the evaluation criteria described below. The results are shown as average values (Table 5).

<Evaluation Criteria>
0: Feeling of sound sleep did not change by the test.
1: Feeling of sound sleep was improved to some extent by the test.
2: Feeling of sound sleep was improved by the test.

| (Composition 4) | (mg) |
|---|---|
| Astaxanthin | 1.5 |
| Zinc yeast (beer yeast containing | 20 |
| 10% by mass zinc) | |
| Ascorbic acid | 20 |
| Thioctic acid10 | |
| Coenzyme Q10 | 10 |
| Selenium yeast (beer yeast containing | 6 |
| 0.2% by mass selenium) | |
| Grape seed extract | 7 |
| Crystalline cellulose | 100 |
| Calcium stearate | 2 |
| Vitamin E | 0.05 |
| γ-Cyclodextrin | 50 |

2. Results

As shown in Table 5, the Example sample showed an evident sleep-improving effect.

TABLE 5

| | Sample used | Evaluation of sleep |
|---|---|---|
| Example 5 | Composition 4 | 1.7 |
| Comparative | Material for | 0.3 |
| Example 5 | Comparative Example | |

### Example 6, Comparative Examples 6 to 8

### 1. Methods

### (i) Animals Used

C57BL/6 mice (male; 8 weeks old; body weight: 22-26 g) were purchased from SLC.

### (ii) Method of Keeping Mice

The mice were individually maintained at a constant temperature (22±2° C.) and humidity (50±2%) in acrylic cages placed in a sound-proof chamber. The mice were kept under a light/dark cycle of 12/12 hours (the time of the beginning of the light period, 7:00 AM), and fed with solid feed for mice (feed name: Labo MR Stock). The mice were provided with the food and water ad libitum.

(iii) Measurement of Amount of Actions

The mice were allowed to recover in a recovery chamber for 4 days. The mice were then transferred to a recording chamber to allow acclimatization for 3 days. The amount of actions was recorded using a sensor which detects infrared radiation from animals (manufactured by Biotex Japan) and Biotex 16CH Act Monitor BAI2216 software (manufactured by Biotex Japan).

This sensor has a detection area expanding at a radiation angle of 90° . This area was divided into 64 (8×8) areas, and the number of times of passage through the areas by each animal was counted as the amount of actions. The amount of actions was measured during the 24 hours before administration.

(iv) Preparation and Administration of Samples

Each of compositions containing the materials described in Table 6 (Example 6, Comparative Examples 7 and 8) was prepared as an Example sample or Comparative Example sample. In Comparative Example 6, purified water was used as a Comparative Example sample. The value for each ingredient of the compositions in Table 6 is expressed in milligrams. The percentages of zinc in the beer yeast and baker's yeast are expressed in percent by mass.

According to Table 6, the Example sample or Comparative Example sample was orally administered to mice at a dose of 10 g/kg using a sonde needle. The administration was carried out by administering the Example sample or Comparative Example sample to the mice at 19:00 (the time of the beginning of the dark period) (n=7-8).

(v) Recording of Amount of Actions

After the oral administration, the cages were replaced in order to give stress to the mice, and the amount of actions were measured for 12 hours thereafter while the number of times of actions per hour was recorded, to calculate the cumulative amount of actions over 6 hours. Subsequently, 24 hours of observation was carried out.

2. Results

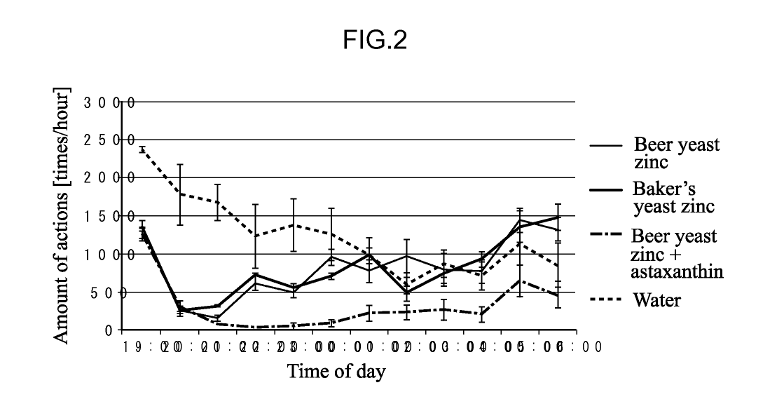

The number of times of actions (cumulative amount of action) over 12 hours after administration was as shown in Table 6 and FIG. 2.

In the Example, a significant decrease in the amount of actions relative to the Comparative Examples was found at the dose of 10 g/kg.

TABLE 6

| | | Comparative | Comparative | Comparative |
|---|---|---|---|---|
| | Example 6 | Example 6 | Example 7 | Example 8 |
| Astaxanthin | 1.25 | — | — | — |
| Zinc yeast | 20 | — | — | 20 |
| (beer yeast | | | | |
| containing | | | | |
| 10% zinc) | | | | |
| Zinc yeast | — | — | 40 | — |
| (baker's yeast | | | | |
| containing | | | | |
| 5% zinc) | | | | |
| Purified water | 250 | 250 | 250 | 250 |
| Cumulative | 1850 | 9900 | 4100 | 4300 |
| amount of | | | | |
| actions | | | | |
| (number of times) | | | | |

### Comparative Examples 9 and 10

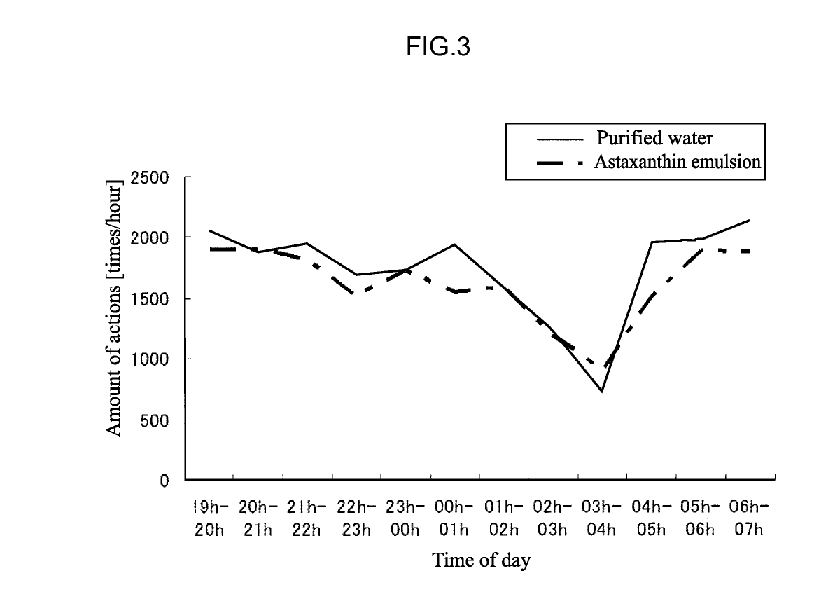

The number of times of actions in mice over 12 hours after administration was measured in the same manner as in Example 6 except that a compound prepared as follows (Comparative Example 9) or purified water (Comparative Example 10) was used. The results are shown in FIG. 3.

<Composition>

### (Ingredients)

| | |
|---|---|
| (1) Haematococcus algae pigment | 2.8 |
| (astaxanthin content: 20% by mass) | |
| (2) Mixed tocopherol | 0.7 |
| (3) Sucrose laurate | 2.6 |
| (4) Polyglyceryl-10 laurate | 0.8 |
| (5) Lecithin | 0.7 |
| (6) Inulin | 12.0 |
| (7) Purified water | 80.4 |

Haematococcus algae pigment (astaxanthin content: 20% by mass) (ASTOTS-S, manufactured by Takeda Shiki Co., Ltd.)

Mixed tocopherol (Riken E Oil 800, manufactured by Riken Vitamin Co., Ltd.)

Sucrose laurate (RYOTO Sugar Ester L-1695, manufactured by Mitsubishi-Kagaku Foods Corporation)

Polyglyceryl-10 laurate (NIKKOL Decaglyn 1-L, manufactured by Nikko Chemicals Co., Ltd.)

Lecithin (Lecion P, manufactured by Riken Vitamin Co., Ltd.)

Inulin (Fuji FF, manufactured by Fuji Nihon Seito Corporation)

(A) The ingredients (1) and (2) were weighed in a container, and mixed under heat with stirring in an incubator at 70° C. After confirming that the mixture was sufficiently mixed, the mixture was kept at 70° C. to obtain a mixture A.

(B) The ingredients (3) to (7) were weighed in a container, and mixed under heat with stirring in an incubator at 70° C. After confirming that the mixture was sufficiently mixed, the mixture was kept at 70° C. to obtain a mixture B.

(C) The mixture A was mixed with the mixture B, and the resulting mixture was uniformly emulsified. As an emulsifier, a homogenizer (manufactured by SMT Corporation) was used for stirring at a rotation speed of 10,000 for 5 minutes, to obtain a mixture C.

(D) The mixture C was subjected to an emulsification operation using a high-pressure homogenizer (Ultimizer HJP-25003, manufactured by Sugino Machine Limited) at a pressure of 240 MPa at a liquid temperature of 45° C., to obtain an astaxanthin emulsion.

The obtained astaxanthin emulsion was applied to a spray drier (ADL310, manufactured by Yamato Scientific Co., Ltd.) at a rate of 10 mL/minute while spray drying was carried out by sending air at 140° C., to prepare a powder of astaxanthin nanoemulsion.

As shown in FIG. 3, it could be confirmed that there was no difference in the amount of actions between the case where the composition which contains astaxanthin but does not contain zinc was used and the case where purified water was used.

### Example 7, Comparative Examples 11 and 12

### 1. Methods

### (i) Animals Used

C57BL/6 mice (male; 12 weeks old; body weight: 24-17 g) were purchased from SLC.

### (ii) Method of Keeping Mice

The mice were individually maintained at a constant temperature (22±2° C.) and humidity (50±2%) in acrylic cages placed in a sound-proof chamber. The mice were kept under a light/dark cycle of 12/12 hours, and fed with solid feed for rats (feed name: Labo MR Stock). The mice were provided with the food and water ad libitum.

(iii) Operation for Placement of Electrodes for Measuring Brain Waves and Myogenic Potential, and Connection of Electrodes to Measuring Apparatus

The mice were subjected to an operation for placement of electrodes for measuring brain waves and the myogenic potential, and then allowed to recover in a recovery chamber for 7 days. Subsequently, the mice were transferred to a record chamber, and measurement cables were connected to the electrodes, followed by allowing acclimatization of the mice for 4 days.

(iv) Preparation and Administration of Samples

Each of compositions containing the materials described in Table 7 (Example 7, Comparative Examples 11 and 12) was prepared as an Example sample or Comparative Example sample, and orally administered to mice at a dose of 10 g/kg using a sonde needle. The administration was carried out by administering the Example sample or Comparative Example sample to the mice at 16:00 (the time of the beginning of the dark period) (n=2). Each value in Table 7 is expressed in milligrams.

(v) Recording and Analysis of Brain Waves and Myogenic Potential

The brain waves and myogenic potential were recorded after amplification (brain waves, 0.5-30 Hz; myogenic potential, 20-200 Hz) and subsequent digitization at a sampling rate of 128 Hz. The analysis was carried out using brain-wave recording software “SleepSign” (manufactured by Kissei Comtec Co., Ltd.). Data obtained during 10 seconds were defined as 1 epoch, and each epoch was automatically judged as awakening, non-REM sleep, or REM sleep based on the frequency components and waveforms of the brain waves and myogenic potential. The brain wave data obtained over 4 hours after administration were analyzed to calculate the durations of awakening, non-REM sleep, and REM sleep per hour. In addition, the length of time required for occurrence of non-REM sleep was measured.

TABLE 7

| | | Comparative | Comparative |
|---|---|---|---|
| | Example 7 | Example 11 | Example 12 |
| Composition prepared in | 6 | — | — |
| Comparative Example 9 | | | |
| (containing 20% by | | | |
| mass astaxanthin) | | | |
| Zinc gluconate | 8 | 8 | — |
| Soybean oil | 250 | 250 | 250 |

2. Results

The durations of non-REM sleep, REM sleep, and awakening over 4 hours after administration are shown in Table 8.

In Example 7, a better effect to increase the duration of non-REM sleep relative to Comparative Examples 11 and 12 was found at the dose of 10 g/kg.

In addition, in Example 7, shortening of the length of time before falling asleep could be found. Each value in Table 8 is expressed in minutes.

TABLE 8

| | Duration of | Duration of | Duration of |
|---|---|---|---|
| | non-REM | REM sleep | awakening |
| | sleep (minutes) | (minutes) | (minutes) |
| Example 7 | 60 | 120 | 10 |
| Comparative | 40 | 140 | 30 |
| Example 11 | | | |
| Comparative | 30 | 150 | 60 |
| Example 12 | | | |

By the invention, an excellent sleep-improving agent, especially an effect to increase the duration of non-REM sleep, can be obtained. In addition, shortening of sleep onset latency and reduction of interruption of sleep can be achieved, and a sedating effect and relaxation effect can be obtained. Thus, a satisfying feeling of sound sleep can be obtained at the time of awakening.

The disclosure of Japanese Patent Application No. 2012-221752 is hereby incorporated by reference.

All publications, patent applications, and technical standards mentioned in this specification are herein incorporated by reference to the same extent as if each individual publication, patent application, or technical standard was specifically and individually indicated to be incorporated by reference.

1. A sleep-improving agent comprising, as active ingredients, a lipid-soluble antioxidant and a divalent metal.

2. The sleep-improving agent according to claim 1, wherein the lipid-soluble antioxidant is a carotenoid.

3. The sleep-improving agent according to claim 1, wherein the lipid-soluble antioxidant is at least one selected from the group consisting of astaxanthin and derivatives thereof.

4. The sleep-improving agent according to claim 1, wherein the lipid-soluble antioxidant is contained in a powder composition obtained by drying an emulsion composition comprising: (a) at least one selected from the group consisting of a sucrose fatty acid ester and a polyglycerol fatty acid ester; and (b) a phospholipid, wherein (a) and (b) have an equal content ratio by mass, or (a) has a higher content ratio by mass.

5. The sleep-improving agent according to claim 1, wherein the divalent metal is incorporated in a yeast.

6. The sleep-improving agent according to claim 1, wherein the divalent metal is zinc.

7. The sleep-improving agent according to claim 1, wherein the mass ratio of the lipid-soluble antioxidant and the divalent metal is from 1:0.01 to 1:10.

8. The sleep-improving agent according to claim 1, further comprising a water-soluble antioxidant.

9. The sleep-improving agent according to claim 8, wherein the water-soluble antioxidant is at least one selected from the group consisting of ascorbic acid, derivatives thereof, and thioctic acid.

10. The sleep-improving agent according to claim 8, wherein the mass ratio of the water-soluble antioxidant and the lipid-soluble antioxidant is from 1:0.01 to 1:10.

11. The sleep-improving agent according to claim 1, wherein the lipid-soluble antioxidant is astaxanthin, and the divalent metal is zinc.

12. The sleep-improving agent according to claim 11, wherein the lipid-soluble antioxidant is astaxanthin, and the lipid-soluble antioxidant is contained in a powder composition obtained by drying an emulsion composition comprising: (a) at least one selected from the group consisting of a sucrose fatty acid ester and a polyglycerol fatty acid ester; and (b) a phospholipid, wherein (a) and (b) have an equal content ratio by mass, or (a) has a higher content ratio by mass.

13. The sleep-improving agent according to claim 12, wherein the lipid-soluble antioxidant is astaxanthin, and the astaxanthin is contained in an astaxanthin-containing oil.

14. A non-REM sleep time-increasing agent comprising, as active ingredients, a lipid-soluble antioxidant and a divalent metal.

15. The non-REM sleep time-increasing agent according to claim 14, wherein the lipid-soluble antioxidant is astaxanthin, and the divalent metal is zinc.

16. The non-REM sleep time-increasing agent according to claim 15, wherein the lipid-soluble antioxidant is astaxanthin, and the lipid-soluble antioxidant is contained in a powder composition obtained by drying an emulsion composition comprising: (a) at least one selected from the group consisting of a sucrose fatty acid ester and a polyglycerol fatty acid ester; and (b) a phospholipid, wherein (a) and (b) have an equal content ratio by mass, or (a) has a higher content ratio by mass.

17. The non-REM sleep time-increasing agent according to claim 16, wherein the lipid-soluble antioxidant is astaxanthin, and the astaxanthin is contained in an astaxanthin-containing oil.

18. A sedative comprising, as active ingredients, a lipid-soluble antioxidant and a divalent metal.
